(19) United States
(12) Patent Application Publication
(10) Pub. No.: US 2005/0019357 A1
(43) Pub. Date: Jan. 27, 2005

(54) (2S,3R,4S)-4-hydroxyisoleucine-containing cosmetic compositions and methods of application

(75) Inventors: Pierre Potier, Paris (FR); Jamal Ouazzani, Massy (FR); Jean-Francois Rodelet, Boulogne Billancourt (FR); Nobumichi Andre Sasaki, Les Ulis (FR); Qian Zhu Wang, Gif-Sur-Yvette (FR)

Correspondence Address: FULBRIGHT & JAWORSKI L.L.P., 600 CONGRESS AVE., SUITE 2400, AUSTIN, TX, 78701, US

(73) Assignees: Caster, Paris (FR); Centre National de la Recherche Scientifique (CNRS), Paris (FR)

(21) Appl. No.: 10/865,723

(22) Filed: Jun. 10, 2004

(30) Foreign Application Priority Data
Jun. 18, 2003 (FR) 03/07358

(51) Int. Cl. A61K 7/00; A61K 31/198

(52) U.S. Cl. 424/401; 514/561

(57) ABSTRACT

The invention relates to a cosmetic composition for topical application containing, in a cosmetically acceptable carrier, an effective quantity of (2S,3R,4S)-4-hydroxyisoleucine and the use of such a composition for combating the natural signs of ageing of the skin and of the scalp.

### BACKGROUND OF THE INVENTION

1. Field of the Invention

The present invention relates to the use of (2S,3R,4S)-4-hydroxyisoleucine in topical application for combating the effects of ageing of the skin and of the scalp.

2. Description of Related Art

(2S,3R,4S)-4-Hydroxyisoleucine is a rare amino acid, absent from humans and animals, which is only found in certain plant species. This amino acid has been known for several years for its insulin-regulating effect and is used as active ingredient in the treatment of diabetes.

### SUMMARY OF THE INVENTION

The applicant has discovered that this same molecule, when administered not internally but by topical application, makes it possible to combat the natural signs of ageing of the skin and of the scalp and in particular androgenogenetic alopecia, or alopecia of the male type.

The subject of the present invention is consequently cosmetic compositions for topical application containing, in a cosmetically acceptable carrier, an effective quantity of (2S,3R,4S)-4-hydroxyisoleucine.

Its subject is also the use of (2S,3R,4S)-4-hydroxyisoleucine for combating the natural signs of ageing of the skin and of the scalp and in particular hair loss linked to ageing.

Its subject is additionally a method for the cosmetic treatment of the skin and of the scalp comprising the application to the skin or the scalp of an effective quantity of (2S,3R,4S)-4-hydroxyisoleucine for a sufficient duration and at a sufficient frequency for the desired cosmetic effect to be obtained.

As indicated above, (2S,3R,4S)-4-hydroxyisoleucine of formula CH₃-CHOH-CHCH₃-CHNH₂-COOH is a very rare natural amino acid which is only found in certain plant species among which there may be mentioned Dioscorea deltoidea, Balanires aegyptiaca, Trigonella foenum-graecum (fenugreek) and Solanum laciniatum. A method for synthesizing this molecule has been described in international patent application WO 01/72688.

The applicant, in the context of a research programme studying the physiological action of 4-hydroxyisoleucine, has discovered, surprisingly, that this molecule caused, at the cellular level, modification of the level of expression of certain genes.

The results of these research studies, obtained by the “cDNA-macroarrays” method and confirmed by quantitative PCR after reverse transcription of the messenger RNAs of epidermal cells (quantitative RT-PCR method) reveal in particular:
a significant underexpression of Cellular Retinoic Acid Binding Protein (CRAB-P-2): this protein has a regulatory role on the quantity of free retinoid acid and its underexpression results in an increase in the availability of retinoic acid at the cellular level;
a significant overexpression of Bullous Pemphigoid Antigen 1 (BPAG-1): this protein is involved in the consolidation of the cytoskeleton and in its cellular cohesion;
a significant overexpression of the JL1-R1 receptor and the decrease in the expression of the TNS & CRAB-2 receptors contribute to the preservation of the equilibrium of the pro-inflammatory and anti-inflammatory effect which results overall in a decrease in inflammation.

Now, the role of retinoic acid in cell renewal, and in particular in the renewal of the cells of the epidermis, on the one hand, and the involvement of the inflammatory phenomena in the degradation of the extra-cellular matrix, on the other hand, are known. From all these biological effects, namely the stimulation of the renewal of epidermal cells, the prevention of the degradation of the extracellular matrix and the strengthening of the cytoskeleton and of the cellular cohesion, the applicant deduced that (2S,3R,4S)-4-hydroxyisoleucine had to have an anti-ageing effect on the epidermis.

### DETAILED DESCRIPTION OF THE INVENTION

The cosmetic compositions of the present invention may be provided in any form allowing easy topical application, for example in the form of a lotion, a serum, a cream, a milk or an ointment.

The concentration of (2S,3R,4S)-4-hydroxyisoleucine in these compositions depends on a number of parameters such as the nature or the seriousness of the cosmetic disorder to be treated (wrinkles, ageing spots, hair loss), the frequency and the duration of application and the possible presence of other cosmetic active ingredients.

The applicant observed that satisfactory results were generally obtained with (2S,3R,4S)-4-hydroxyisoleucine concentrations of between 0.02% and 2% by weight relative to the total weight of the cosmetic composition.

The anti-ageing effect on the epidermis was confirmed in vivo in a study relating to the anti-hair loss properties of (2S,3R,4S)-4-hydroxyisoleucine carried out on volunteer subjects affected by alopecia, having a proportion of hair in the telogen phase greater than or equal to 15%. This study, the conditions of which are described in detail in the exemplary embodiment below, showed that (2S,3R,4S)-4-hydroxyisoleucine was particularly effective when it is used as active ingredient in an anti-hair loss composition already containing a particular combination of anti-hair loss active ingredients. This anti-hair loss composition is described in application FR 2 776 184 of which the applicant is proprietor. It contains an aqueous extract of the mushroom Lentinus edodes rich in 1,3-β-D-1,6-β-D-glucan, procyanidolic oligomers extracted from grapeseed cuticle, and essential oil extracted from the flowers of Cananga odorata.

Consequently in a preferred embodiment of the invention, the cosmetic composition for topical application of the present invention is an anti-hair loss hair composition and in particular an anti-hair loss composition containing, in addition to an effective quantity of (2S,3R,4S)-4-hydroxyisoleucine, an aqueous extract of the mushroom Lentinus edodes rich in 1,3-β-D-1,6-β-D-glucan, procyanidolic oligomers extracted from grapeseed cuticle, and essential oil extracted from the flowers of Cananga odorata.

The obtaining of the desired cosmetic effect requires the repeated application of the active ingredient, in the form of a cosmetic composition as described above, for a sufficient duration and at a sufficient frequency which can be easily determined by persons skilled in the art by virtue of their general technical knowledge. For the obtaining of an anti-hair loss effect, the cosmetic composition according to the invention is preferably applied for a period of at least 12 weeks, in particular of at least 16 weeks, at a frequency preferably at least equal to 1 application every 72 hours, in particular at least equal to 1 application every 48 hours.

### EXAMPLE 1

The anti-hair loss efficacy of (2S,3R,4S)-4-hydroxyisoleucine was tested by incorporating it at a concentration of 0.05% by weight into a commercial anti-hair loss hair composition (Phytoaxil®) containing an aqueous extract of the mushroom Lentinus edodes rich in 1,3-β-D-1,6-β-D-glucan, procyanidolic oligomers extracted from grapeseed cuticle, and essential oil extracted from the flowers of Cananga odorata, and by comparing the results obtained with this new composition to those obtained with the Phytoaxil® composition alone.

This study was carried out on a sample of 25 volunteers for each composition tested. The only people included in the study were men from 20 to 50 years old (average age of each group 37 years) with alopecia, that is to say having a proportion of hair in the telogen phase, determined by videotrichogram, greater than 15%.

The apparatus used is a videomicroscope equipped with a ×25 lens, which is mobile and optical fibre-based, linked to a computer system for image acquisition. The lens is placed directly over the zone to be studied, the image is digitized, recorded and analysed with the aid of a specific software (COUNTHAIR®) developed by the company DERMSCAN which makes it possible to count the hair strands and to measure their length. The parameters evaluated are
a) the hair density,
b) the number and the proportion of hair in the anagen phase (growth phase),
c) the number and the proportion of hair in the telogen phase (growth arrest) and
d) the average diameter of the hair.

The chronological order of the study is the following:
week 0, day 0: shaving of the hair in a defined zone and recording of the first videotrichogram;
week 0, day 2: second videotrichogram, determination of the proportion of hair in the telogen phase and selection of volunteers in whom this proportion is greater than 15%, distribution of the composition to be tested (Phytoaxil® or Phytoaxil® +0.05% of (2S,3R,4S)-4-hydroxyisoleucine) in a sufficient quantity for four weeks of treatment at the rate of one application every two days;
week 4, day 0: shaving of the hair zone, distribution of the composition to be tested in a sufficient quantity for four weeks of treatment at the rate of one application every two days;
week 8, day 0: shaving of the hair zone, videotrichogram;
week 8, day 2: videotrichogram, distribution of the composition to be tested in a sufficient quantity for four weeks of treatment at the rate of one application every two days;
week 12, day 0: shaving of the hair zone, distribution of the composition to be tested in a sufficient quantity for four weeks of treatment at the rate of one application every two days;
week 16, day 0: shaving of the hair zone, videotrichogram,
week 16, day 2: videotrichogram.

At the end of the study, there are thus available the initial proportion of hair in the telogen phase and the proportion of hair in the telogen phase at the end of 8 and 16 weeks.

The statistical evaluation of the results thus obtained for the two groups of volunteer subjects is carried out according to the Student's test on paired data, by calculating the probability p of observing a difference between the proportion of hair in the telogen phase before and after treatment at least as high as that which was really observed, if the null hypothesis was true, that is to say if the treatment had no effect.

If the value of p calculated from the results obtained is less than 5% (0.05), the null hypothesis is rejected and it is concluded that there is a significant difference due to the treatment.

Table 1 below shows the value of p calculated from the results of the measurements of the hair diameter, of the proportion of hair in the telogen phase and of the hair density, carried out on the volunteers in the two groups at the end of 8 weeks of treatment and of 16 weeks of treatment.

TABLE 1
Values of p (Student's test)

| | Phytoaxil ® + 4- | |
|---|---|---|
| | hydroxyisoleucine | Control |
| Diameter of the hair after | 0.260 | 0.070 |
| 8 weeks | | |
| Diameter of the hair after | 0.004 | 0.784 |
| 16 weeks | | |
| Telogen phase after 8 weeks | 0.097 | 0.400 |
| Telogen phase after 16 weeks | 0.004 | 0.862 |
| Hair density after 8 weeks | 0.078 | 0.711 |
| Hair density after 16 weeks | 0.001 | 0.115 |

These results clearly show that at the end of 16 weeks of treatment with the composition according to the invention containing 0.05% by weight of (2S,3R,4S)-4-hydroxyisoleucine, a significant increase in the hair density and in the diameter of the hair, and a significant reduction in the number of hair in the telogen phase, are observed.

### EXAMPLE 2

Anti-hair loss hair lotion

| | |
|---|---|
| (2S,3R,4S)-4-Hydroxyisoleucine | 0.05% |
| Glycerine | 9.00% |
| Dimethyl isosorbide | 6.00% |
| Perfume | 4.50% |
| Solanum tuberosum extract | 1.00% |
| Cananga odorata essential oil | 1.00% |
| Serenoa repens extract | 0.20% |
| Lentinus edodes extract | 0.20% |
| Grape PCO | 0.10% |
| Preservatives | qs |
| Ethanol | 25.00% |
| Distilled water | qs 100% |

### EXAMPLE 3

Anti-hair loss hair serum

| | |
|---|---|
| (2S,3R,4S)-4-Hydroxyisoleucine | 0.05% |
| Ethanol (96% vol.) | 20.00% |
| Glycerine | 5.00% |
| Soya-bean protein hydrolysate | 0.80% |
| Prunus africana oily extract | 0.30% |
| PPG-1-PEG-9 lauryl glycol ether | 5.00% |
| Perfume | qs |
| Preservative (ethylparaben, butylparaben, | qs |
| phenoxyethanol) | |
| Demineralized water | qs 100% |

### EXAMPLE 4

Anti-wrinkle serum for the skin

| | |
|---|---|
| 4-Hydroxyisoleucine | 2.00% |
| PEG-8 caprylic/capric glycerides | 30.00% |
| Polyglyceryl-3 diisostearate | 20.00% |
| Octadecyl myristate | 12.00% |
| Ascorbyl palmitate | 1.00% |
| Pinus pinaster bark extract | 0.20% |
| Perfume | qs |
| Preservative (ethylparaben, butylparaben, | qs |
| phenoxyethanol) | |
| Demineralized water | qs 100% |

### EXAMPLE 5

Anti-wrinkle beauty cream

| | |
|---|---|
| (2S,3R,4S)-4-Hydroxyisoleucine | 10.00% |
| Glycerine | 10.00% |
| Triethanolamine stearate | 10.00% |
| Cetyl alcohol | 0.50% |
| Stearin triple press | 0.50% |
| Sweet almond oil | 5.00% |
| Petroleum jelly | 5.00% |
| Dimethicone | 2.00% |
| Preservative (parabens) | qs |
| Perfume | qs |
| Demineralized water | qs 100% |

### EXAMPLE 6

Anti-spot beauty cream

| | |
|---|---|
| (2S,3R,4S)-4-Hydroxyisoleucine | 0.80% |
| PEG-6 stearate/PEG-32 stearate | 6.50% |
| Self-emulsifying beeswax | 4.50% |
| Fluid liquid paraffin | 4.00% |
| Dimethicone 200/100 | 3.00% |
| PEG-30 castor oil | 1.50% |
| Diethyl trioxopimelate | 0.80% |
| Perfume | qs |
| Preservative (parabens) | qs |
| Demineralized water | qs 100% |

### EXAMPLE 7

Body milk

| | |
|---|---|
| (2S,3R,4S)-4-Hydroxyisoleucine | 0.03% |
| Fluid liquid paraffin | 8.00% |
| Sweet almond oil | 4.00% |
| Self-emulsifying glyceryl stearate | 3.50% |
| Cetyl alcohol | 2.00% |
| Oxyethylenated (20 EO) stearyl alcohol | 1.50% |
| Dimethicone 200/100 | 1.00% |
| Carbomer | 0.30% |
| Menthol | 0.10% |
| Perfume | qs |
| Preservative (parabens) | 0.30% |
| Demineralized water | qs 100% |

1-12. (Canceled)

13. A composition comprising (2S,3R,4S)-4-hydroxyisoleucine in a carrier suitable for topical application.

14. The composition of claim 1, further defined as comprising from 0.02 to 2% by weight of (2S,3R,4S)-4-hydroxyisoleucine.

15. The composition of claim 1, further comprising an aqueous extract from a mushroom Lentinus edodes comprising 1,3-β-D-1,6-β-D-glucan.

16. The composition of claim 1, further comprising procyanidolic oligomers from grapeseed cuticle.

17. The composition of claim 1, further comprising essential oil from the flowers of Cananga odorata.

18. The composition of claim 1, further comprising:
a) an aqueous extract from a mushroom Lentinus edodes comprising 1,3-β-D-1,6-β-D-glucan;
b) procyanidolic oligomers from grapeseed cuticle; and
c) essential oil from the flowers of Cananga odorata.

19. The composition of claim 13, further comprising at least one additional anti-hair loss agent.

20. The composition of claim 1, further defined as a lotion, a serum, a cream, a milk, or an ointment.

21. A method for the treatment of the skin and/or scalp comprising applying to the skin and/or scalp of the subject a composition comprising (2S,3R,4S)-4-hydroxyisoleucine.

22. The method of claim 21, wherein the composition is further defined as comprising from 0.02 to 2% by weight of (2S,3R,4S)-4-hydroxyisoleucine.

23. The method of claim 21, further comprising applying an aqueous extract of the mushroom Lentinus edodes comprising 1,3-β-D-1,6-β-D-glucan to the skin and/or scalp of the subject.

24. The method of claim 21, further comprising applying procyanidolic oligomers extracted from grapeseed cuticle to the skin and/or scalp of the subject.

25. The method of claim 21, further comprising applying essential oil extracted from the flowers of Cananga odorata to the skin and/or scalp of the subject.

26. The method of claim 21, further comprising applying to the skin and/or scalp of the subject:
a) an aqueous extract of the mushroom Lentinus edodes comprising 1,3-β-D-1,6-β-D-glucan;
b) procyanidolic oligomers extracted from grapeseed cuticle; and
c) essential oil extracted from the flowers of Cananga odorata.

27. The method of claim 21, wherein the composition is applied repeatedly during a period of at least 12 weeks.

28. The method of claim 27, wherein the composition is applied during a period of at least 16 weeks.

29. The method of claim 26, wherein the composition is applied repeatedly at a frequency of 1 application at least every 72 hours, for a period of time.

30. The method of claim 29, wherein the composition is applied repeatedly at a frequency of 1 application at least every 48 hours, for a period of time.

31. The method of claim 21, further defined as a method of combating signs of aging of the skin and of the scalp.

32. The method of claim 31, further defined as a method of combating hair loss.

33. The method of claim 32, further comprising applying at least one additional anti-hair loss agent to the skin and/or scalp of the subject.

34. The method of claim 21, wherein the composition is further defined as a lotion, a serum, a cream, a milk, or an ointment.
